(19) United States
(12) Patent Application Publication
(10) Pub. No.: US 2014/0155780 A1
(43) Pub. Date: Jun. 5, 2014

(54) BODY FLUID COLLECTION DEVICE

(71) Applicant: Sysmex Corporation, Kobe-shi Hyogo (JP)

(72) Inventors: Yasuo KIKKAWA, Kobe-shi (JP); Mina KAMEI, Kobe-shi (JP); Akihito TAKEZAKI, Kobe-shi (JP)

(73) Assignee: Sysmex Corporation, Kobe-shi Hyogo (JP)

(21) Appl. No.: 14/095,432

(22) Filed: Dec. 3, 2013

(30) Foreign Application Priority Data
Dec. 4, 2012 (JP) 2012-265250

(51) Int. Cl. A61B 10/00

(52) U.S. Cl. CPC A61B 10/0045
USPC 600/573

(57) ABSTRACT

A body fluid collection device for collecting body fluid through the skin, comprising: a sheet-like substrate provided with an adhesive surface; a first collection body for collecting body fluid, provided on the adhesive surface; a first detachable sheet which is detachably adhered on the adhesive surface so as to cover the first collection body; and a second detachable sheet which is detachably adhered on the adhesive surface, wherein one end of the second detachable sheet in a predetermined direction is overlaid on the far end of the first detachable sheet in the predetermined direction, and part of the far end of the first detachable sheet is separated from the adhesive surface in a non-adhered state is disclosed.

### RELATED APPLICATIONS

This application claims priority under 35 U.S.C. §119 to Japanese Patent Application No. 2012-265250 filed on Dec. 4, 2012, the entire content of which is hereby incorporated by reference.

### FIELD OF THE INVENTION

The present invention relates to a fluid collection device. More specifically, the present invention relates to a body fluid collection device to be used by attaching to the skin when collecting body fluids such as interstitial fluid extracted from the skin of a living body for performing various further analyses.

### BACKGROUND OF THE INVENTION

Conventional body fluid collection members are known to form micropores in the skin of a subject and adhere a collection member having a gel-like collection body on the area in which the micropores are formed to extract interstitial fluid through the skin (for example, Japanese Laid-Open Patent Application No. 2011-227042). The collection body adhered to the adhesive layer of the sheet body is covered by a monolayer detachable liner in the collection member disclosed in Japanese Laid-Open Patent Application No. 2011-227042. The collection member is used while adhered to the skin by removing the detachable liner and then positioning the collection body on a predetermined region of the skin.

Moreover, Japanese Utility Model H6-61226 discloses a percutaneous absorption plaster having a pad 3 covered by dual layers of detachable cover sheets 5 and 6, and a pad 4 containing a drug agent adhered to an adhesive surface 2 of the adhesive sheet 3. In the percutaneous absorption plaster disclosed in Patent Document 2, the dual layer detachable sheets 5 and 6 are mutually overlaid at the center of the pad 4.

In the collection member disclosed in Japanese Laid-Open Patent Application No. 2011-227042, a problem arises in that the detachable liner is difficult to peel off when being used compared to providing a dual layer detachable liner since only a monolayer detachable liner is used.

In the percutaneous absorption plaster of Japanese Laid-Open Patent Application No. 2011-227042, there is concern that the pad 4 may become contaminated when fingers touch the pad peeling off the detachable paper on the lower side after the detachable paper has been peeled away on the upper side since the detachable sheets 5 and 6 overlap in the center of the pad 4.

In view of the above information, an object of the present invention is to provide a body fluid collection device in which a detachable liner can be easily peeled off without touching the collection body.

### SUMMARY OF THE INVENTION

The scope of the present invention is defined solely by the appended claims, and is not affected to any degree by the statements within this summary.

A first aspect of the present invention is a body fluid collection device for collecting body fluid through the skin, comprising:

a sheet-like substrate provided with an adhesive surface;

a first collection body for collecting body fluid, provided on the adhesive surface;

a first detachable sheet which is detachably adhered on the adhesive surface so as to cover the first collection body; and

a second detachable sheet which is detachably adhered on the adhesive surface, wherein

one end of the second detachable sheet in a predetermined direction is overlaid on the far end of the first detachable sheet in the predetermined direction, and

part of the far end of the first detachable sheet is separated from the adhesive surface in a non-adhered state.

A second aspect of the present invention is a body fluid collection device for collecting body fluid through the skin, comprising:

a sheet-like substrate provided with an adhesive surface;

a first collection body for collecting body fluid, provided on the adhesive surface;

a first detachable sheet which is detachably adhered on the adhesive surface so as to cover the first collection body; and

a second detachable sheet which is detachably adhered on the adhesive surface, wherein

one end of the second detachable sheet in a predetermined direction is overlaid on the far end of the first detachable sheet in the predetermined direction,

the first collection body has a thickness of 0.05 mm or greater but less than 1.5 mm,

the first detachable sheet has a thickness of 0.025 mm or greater but less than 0.100 mm, and is made of at least one in a group of polyethylene terephthalate and polyolefin, and

the distance between the far end of the first detachable sheet in the predetermined direction and the far end of the first collection body in the predetermined direction is 1 mm or greater but less than 3 mm.

A third aspect of the present invention is a body fluid collection device for collecting body fluid through the skin, comprising:

a sheet-like substrate provided with an adhesive surface;

a first collection body for collecting body fluid, provided on the adhesive surface;

a second collection body for collecting body fluid, provided on the adhesive surface;

a first detachable sheet which is detachably adhered on the adhesive surface so as to cover the first collection body; and

a second detachable sheet which is detachably adhered on the adhesive surface so as to cover the second collection body; wherein

the first collection body and the second collection body are provided so as to be mutually separated along the predetermined direction on the adhesive surface, and

one end of the second detachable sheet in a predetermined direction is overlaid on the far end of the first detachable sheet in the predetermined direction, that is, part of the far end of the first detachable sheet is separated from the adhesive surface in a non-adhered state.

### Effect of the Invention

The body fluid collection device of the present invention allows the detachable liners to be easily peeled away without touching the collection bodies.

### BRIEF DESCRIPTION OF THE DRAWINGS

FIG. 1 is a perspective view showing examples of the body fluid collection device of the present invention and piercing device used therewith;

FIG. 2 is a perspective view of the fine needle tip installed on the piercing device shown in FIG. 1;

FIG. 3 is a cross section view illustration of the skin in which micropores have been formed by the piercing device;

FIG. 4 is a plan view of an embodiment of the body fluid collection device of the present invention;

FIG. 5 is an exploded perspective view of the body fluid collection device of FIG. 4;

FIG. 6 is a cross sectional view of the body fluid collection device of FIG. 4;

FIGS. 7(a) through 7(d) illustrate the method of use of the body fluid collection device;

FIG. 8 is an exterior perspective view of a biological component analyzer.

FIG. 9 illustrates the principle of perspiration measurement using the first collection body;

FIG. 10 is a plan view of the perspiration collection device of another embodiment of the present invention;

FIG. 11 is an exploded perspective view of the perspiration collection device of FIG. 10;

FIG. 12 is a cross sectional view of the perspiration collection device of FIG. 10;

FIG. 13 is a plan view of the interstitial fluid collection device of another embodiment of the present invention;

FIG. 14 is an exploded perspective view of the interstitial fluid collection device of FIG. 13;

FIG. 15 is a cross sectional view of the interstitial fluid collection device of FIG. 13;

FIGS. 16(a) through 16(d) illustrate the procedure of adhering the perspiration collection device on the skin S of a subject; and

FIGS. 17(a) through 17(d) illustrate the procedure of adhering the interstitial fluid collection device on the skin S of a subject.

### DETAILED DESCRIPTION OF THE PREFERRED EMBODIMENTS

The embodiments of the body fluid collection device of the present invention are described in detail hereinafter with reference to the accompanying drawings.

The body fluid collection device of the present invention is used to collect interstitial fluid through micropores formed in the skin, and the piercing device for forming these micropores is first described below.

Piercing Device

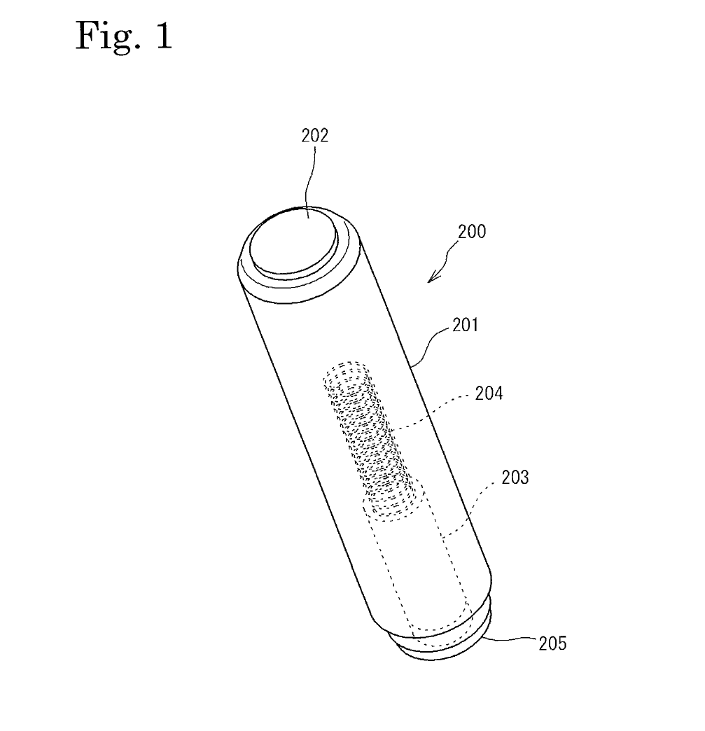

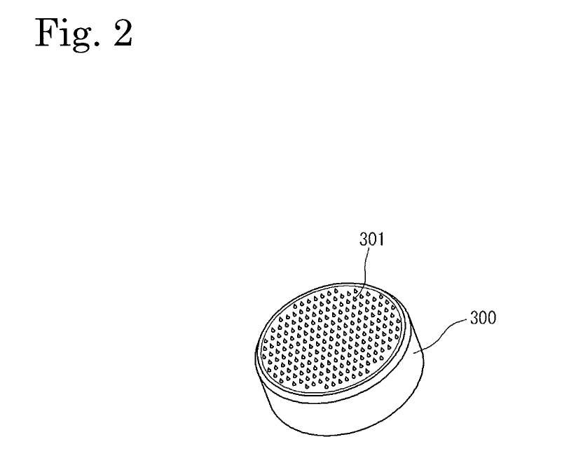

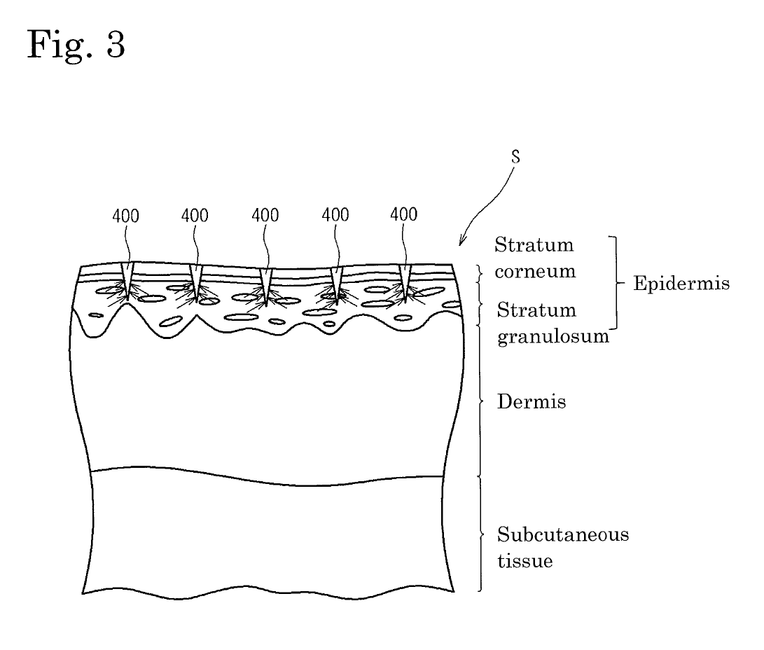

FIG. 1 is a perspective view showing examples of the piercing device 200, which is a micropore forming device, used to form micropores in the skin for the extraction of interstitial fluid through the skin; FIG. 2 is a perspective view of a fine needle tip 300 installed on the piercing device 200 shown in FIG. 1; and FIG. 3 is a cross sectional view of the skin S in which micropores have been formed by the piercing device 200.

As shown in FIGS. 1 through 3, the device forms extraction holes (micropores 400) in the skin S of a subject by installing the piercing device 200 on the sterilized fine needle tip 300, and abutting the fine needles 301 of the fine needle tip 300 against the skin (skin S of the subject) of the living body. The fine needles 301 of the fine needle tip 300 are sized so that the micropores 400 extend into the epidermis of the skin S but do not reach as far as the dermis.

As shown in FIG. 1, the piercing device 200 has a cylindrical case 201, release button 202 provided on the top end surface of the case 201, and an array chuck 203 and spring member 204 provided inside of the case 201. An opening (not shown in the drawings), which allows the fine needle tip 300 to pass through, is formed in the bottom end surface (surface which contacts the skin) of the skin contact part 205 at the bottom end of the case 201. The spring member 204 has the function of exerting a force on the array chuck 203 in the piercing direction. The array chuck 203 can have the fine needle tip 300 installed at the bottom end. A plurality of fine needles 301 are formed on the bottom surface of the fine needle tip 300. The bottom surface of the fine needle tip 300 has a circular shape. The piercing device 200 has a locking mechanism (not shown in the drawings) which locks the array chuck 203 in a state of being pressed upward (reverse of the piercing direction) against the force exerted by the spring member 204, and the locking mechanism is configured to release the hold on the array chuck 203 when the user (subject) presses the release button 202, whereupon the array chuck 203 moves in the piercing direction via the force exerted by the spring member 204 and the fine needle tip 300 is pushed through the opening allowing the fine needles 301 to pierce skin.

### Body Fluid Collection Device

The body fluid collection device is described below in terms of adhering the device to the micropore region formed in the skin S of the subject using the previously mentioned piercing device 200, and collecting the body fluid extracted through the micropores.

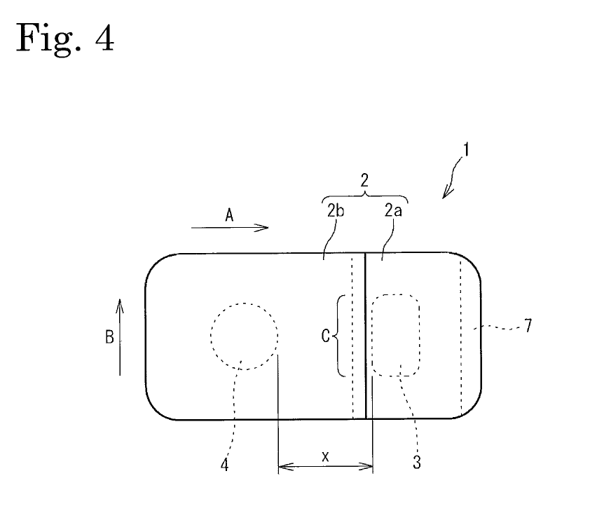

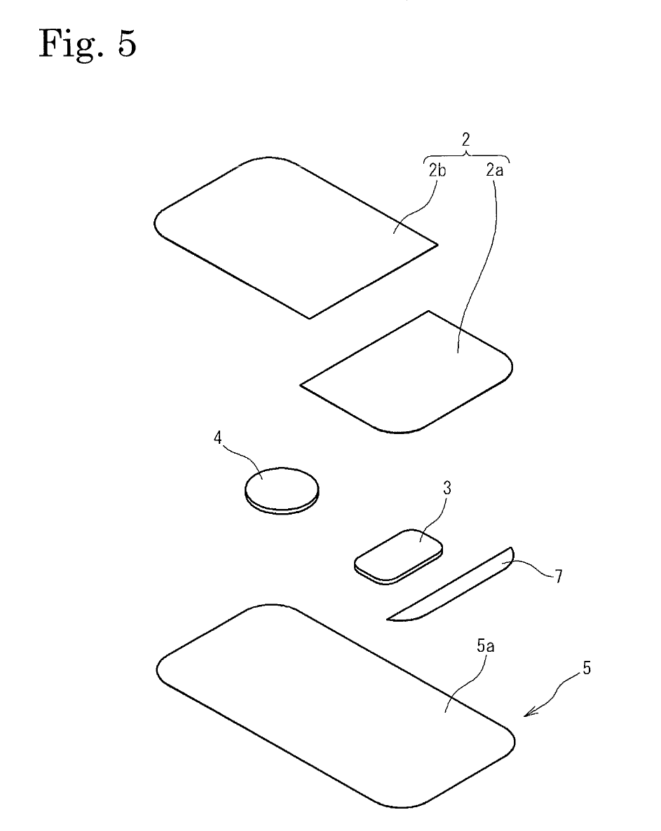

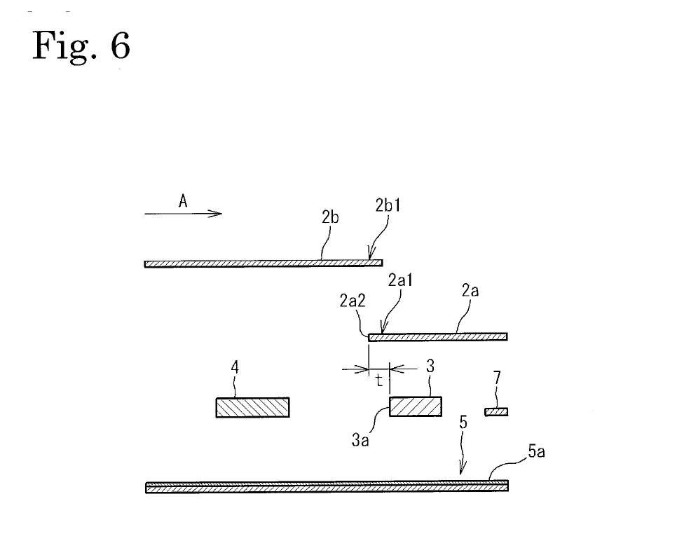

FIG. 4 is a plan view of an embodiment of the body fluid collection device 1 of the present invention, FIGS. 4 and 5 are exploded perspective views of the body fluid collection device 1 of FIG. 4, and FIG. 6 is a cross sectional illustration of the body fluid collection device 1 of FIG. 4. Note that elements such as the sheets and the like which configure the body fluid collection device 1 of the embodiment of the present invention are extremely thin members, the dimensions of which in the width direction are exaggeratedly drawn for ease of understanding in FIG. 6.

The body fluid collection device 1 is configured by a detachable sheet 2, first and second collection bodies 3 and 4, and a substrate 5 sequentially from the side which is adhered to the skin S of the subject (top side in FIGS. 5 and 6). Each of these elements is described below.

### Detachable Sheet

The detachable sheet is configured as a two-ply sheet, that is, a first detachable sheet 2a and a second detachable sheet 2b. The first detachable sheet 2a is detachably adhered to the adhesive surface of the substrate 5 so as to cover the first collection body 3, and the second detachable sheet is detachably adhered to the adhesive surface of the substrate 5 so as to cover the second collection body 4.

The first detachable sheet 2a and the second detachable sheet 2b are substantially rectangular in shape, and made of a synthetic resin such as polyethylene terephthalate (PET), or polyolefin. The thickness of the first detachable sheet 2a and the second detachable sheet 2b is not particularly limited in the present invention, and is normally about 0.025 to 0.100 mm.

A silicone coating (silicone process) is provided on the surface to be adhered (the surface to be applied on the adhesive surface of the substrate 5) of the first detachable sheet 2a and the second detachable sheet 2b so that the detachable sheet can be removed from the adhesive surface.

### Collection Body

The first collection body 3 and the second collection body 4 are disposed in mutual separation along the longitudinal direction, that is, the predetermined direction (A direction in FIGS. 4 and 6), of the substrate 5 on the adhesive surface of the rectangular substrate 5 which has four beveled corners.

The first collection body 3 has a rectangular shape and four beveled corners, and is positioned on the skin S of a subject in the region in which micropores are not formed. The second collection body 4, however, has a circular shape and is positioned in the region in which micropores have been formed in the skin S of the subject. In the present embodiment, the size of the first collection body 3 is 7.1×12.2 mm with a thickness of 0.7 mm. The diameter of the second collection body 4 is 10 mm, and the thickness is 0.7 mm. The shape of the first collection body 3 is not specifically limited to rectangular inasmuch as circular, and square shapes are also possible. The shape and size of the second collection body 4 is not specifically limited in the present invention, insofar as the shape and size conforms to the shape and size of the micropore region formed in the skin S. From the perspective of easily distinguishing the collected body fluid, the shape of the first collection body 3 is preferably different than the shape of the second collection body 4. From the perspective of easy disposition on the micropore region, the shape of the second collection body preferably corresponds to the shape of the micropore region formed in the skin S. Although nor specifically limited in the present invention, the thickness of the first collection body 3 and the second collection body 4 is normally selected from a range from 0.05 to 1.5 mm since these bodies must be covered by the detachable sheet.

The first collection body 3 is used to collect perspiration from the region of the skin S in which micropores are not formed. The second collection body 4, however, is used to collect interstitial fluid from the region of the skin S in which micropores are formed. The first collection body 3 is a member which collects perspiration from the region of the skin in which micropores are not formed in order to determine and correct the reliability of the analysis results without performing a measurement using the interstitial fluid collected by the second collection body 4.

The first collection body 3 and the second collection body 4 are gels with water retention characteristics capable of retaining interstitial fluid extracted from the skin of the subject, and contain purified water as an extraction medium. The gel is not specifically limited in the present invention insofar as the gel is capable of collecting perspiration and interstitial fluid, and preferred gels will include at least one type of hydrophilic polymer selected from a group which includes polyvinylpyrrolidone and polyvinyl alcohol. The hydrophilic polymer forming the gel may be a polyvinyl alcohol alone or polyvinylpyrrolidone alone, or a mixture thereof, and more preferably is polyvinyl alcohol alone, or a mixture of polyvinylalcohol and polyvinylpyrrolidone.

The gel may be formed by methods which bridge hydrophilic polymers in an aqueous solution. The gel may be formed by methods which form a thin film of aqueous solution of hydrophilic polymer on a substrate wherein the hydrophilic polymer contained in the film coating is bridged. Although chemical bridging methods and radiation bridging methods may be used as the hydrophilic polymer bridging method, use of a radiation bridging method is preferable since contamination of the chemical compounds in the gel by impurities is unlikely.

The distance x (refer to FIG. 4) between the far end of the first collection body 3 in the predetermined direction (A direction) and the far end of the second collection body 4 in the predetermined direction (A direction) is not specifically limited in the present invention, however, the distance X is normally 3 to 20 mm since the condition of the skin S changes when the second collection body 4 is too excessively separated from the skin S of the subject considering the use of the first collection body 3 for measurement value correction and the like, and preferably 10 to 20 mm considering the ease of handling the body fluid collection device 1.

### Substrate

The substrate 5 bearing the first collection body 3 and the second collection body 4 has a rectangular shape with four beveled corners, and one side surface (surface on the side bearing the first collection body 3 and the second collection body 4) is an adhesive surface 5a which is coated with an adhesive such as an acrylic adhesive.

The substrate 5 may be made of synthetic resin, such as polyethylene, polypropylene, polyester, or polyurethane. The thickness of the substrate 5 is not specifically limited in the present invention, and is normally about 0.005 to 0.200 mm.

As shown in FIGS. 4 and 6, one end 2b1 of the second detachable sheet 2b in the predetermined direction (A direction) which covers the second collection body 4 is superimposed over the far end of the first detachable sheet 2a which covers the first collection body 3, in the present embodiment. Part of the far end 2a1 of the first detachable sheet 2a is separated from the adhesive surface 5a of the substrate 5 in a non-adhered state. More specifically, the far end 2a1 of the first detachable sheet 2a is not adhered completely to the entire adhesive surface 5a of the substrate 5 in the width direction (B direction in FIG., 4) of the substrate 5, and the center part C in the width direction (refer to FIG. 4) is separated from the adhesive surface 5a of the substrate 5 in a non-adhered state. In other words, the center part in the width direction of the far end 2a1 of the first detachable sheet 2a is separated from the adhesive surface 5a of the substrate 5 so as to form a space between the center part and the adhesive surface 5a. Hence, the adhesion force of the far end 2a1 of the first detachable sheet 2a relative to the adhesive surface 5a is controlled or inhibited. As a result, the first detachable sheet 2a can be easily peeled away from the substrate 5 to adhere the body fluid collection device 1 to the skin S of a subject as shall be described later.

The distance t between the far end 2a2 of the first detachable sheet 2a in the predetermined direction and the far end 3a of the first collection body 3 is set at a distance that maintains the “non-adhered state” as previously mentioned. The distance t differs depending on the quality and thickness of materials of the first detachable sheet 2a and the thickness of the first collection body 3, however, it has been confirmed by experiment that a distance t shorter than the distance L defined by the equation (1) below is preferable when the adhesion force of the adhesive surface 5a of the substrate 5 is 4.0 N/20 mm using polyethyleneterephthalate (silicone coating on the surface to be bonded) at a thickness of 75 μm as the first detachable sheet 2a. Where d is the thickness of the first collection body 3.

L=2.4/0.7×d (1)

The specific range of the distance t is usually 1 to 3 mm when the adhesion force of the adhesive surface 5a of the substrate 5 is 4.0 N/20 mm using, for example, polyethyleneterephthalate having a thickness of 75 μm as the first detachable sheet 2a.

In the present embodiment, a strip-like knob 7 is provided along the short side of the first collection body 3 of the adhesive surface 5a of the substrate 5. The knob 7 is a member having a thickness of approximately 0.005 to 1 mm made of a synthetic resin such as polyethyleneterephthalate, polypropylene, polyester, and polyurethane and the like. The first detachable sheet 2a has a shape which covers the knob 7, but the part which covers the knob 7 is in a non-adhered state, since this part is not in contact with the adhesive surface 5a of the substrate 5.

The surface (bottom side surface in FIG. 6) of one side of the knob 7 is adhered to the adhesive surface 5a of the substrate 5 so that the other surface is in contact with only the first detachable sheet 2a. Therefore, when the detachable sheet 2 is peeled from the substrate 5 and the substrate 5 is adhered to the skin S of a subject according to a procedure described later, the far surface of the knob 7 which is in contact with the skin S is not adhered to the skin S. As a result, the substrate 5 can be simply withdrawn from the skin S after body fluid collection by using the knob 7.

### Interstitial Fluid Collection Method

The method of collection of interstitial fluid and perspiration using the above mentioned body fluid collection device 1 is described below. The skin S of the subject is first washed with alcohol to eliminate substances (perspiration, dirt and the like) which may disturb the measurement results, then micropores are formed in the skin by the piercing device 200 with the installed fine needle tip 300.

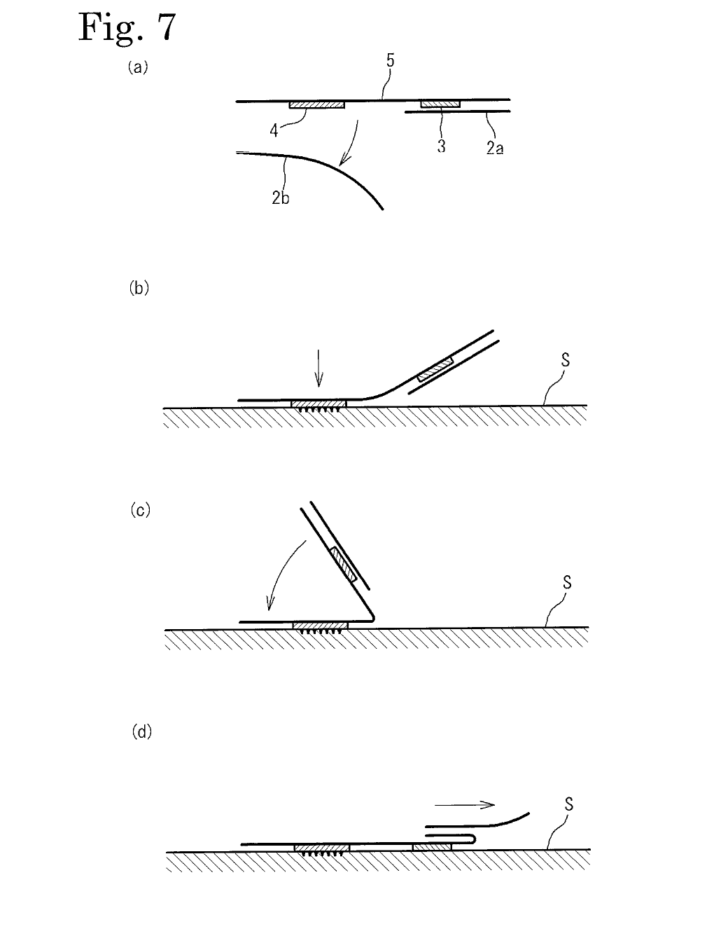

The body fluid collection device 1 is then adhered to the skin S of the subject. FIG. 7 illustrates the procedure of adhering the body fluid collection device 1 on the skin S of a subject. The second detachable sheet 2b is first peeled from the substrate 5 (procedure (a)). The adhesive surface 5a of the substrate 5 which was exposed by peeling away the second detachable sheet 2b is then adhered to the skin S of the subject by positioning the second collection body 4 on the region of the skin S in which micropores were formed (procedure (b)). Thereafter, the first detachable sheet 2a is folded over on the second collection body 4 side (procedure (c)), the first detachable sheet 2a is slid from the folded state so as to press on the skin S of the subject and gradually adhere the adhesive surface 5a which is covered by the first detachable sheet 2a on the skin S of the subject (procedure (d)). The body fluid collection device 1 is adhered to the skin S of the subject through this sequence of procedures.

In the body fluid collection device 1 of the present embodiment, the detachable sheet is formed by two layers of detachable sheets (first detachable sheet 2a, second detachable sheet 2b), and one end 2b1 of the second detachable sheet is overlaid on the far end 2a1 of the first detachable sheet 2a. Part of the far end 2a1 of the first detachable sheet 2a is separated from the adhesive surface 5a of the substrate 5 in a non-adhered state. As described previously, the second detachable sheet 2b therefore is first peeled off to adhere the exposed adhesive surface 5a to the skin, then the first detachable sheet 2a is folded back and the first detachable sheet 2a is slid along the skin surface to easily adhere the body fluid collection device on the skin of the subject without touching the first and second collection bodies 3 and 4.

When a predetermined time (for example, 180 minutes) has elapsed after the body fluid collection device 1 is adhered to the skin S of the subject, the body fluid collection device 1 is peeled away from the skin S of the subject.

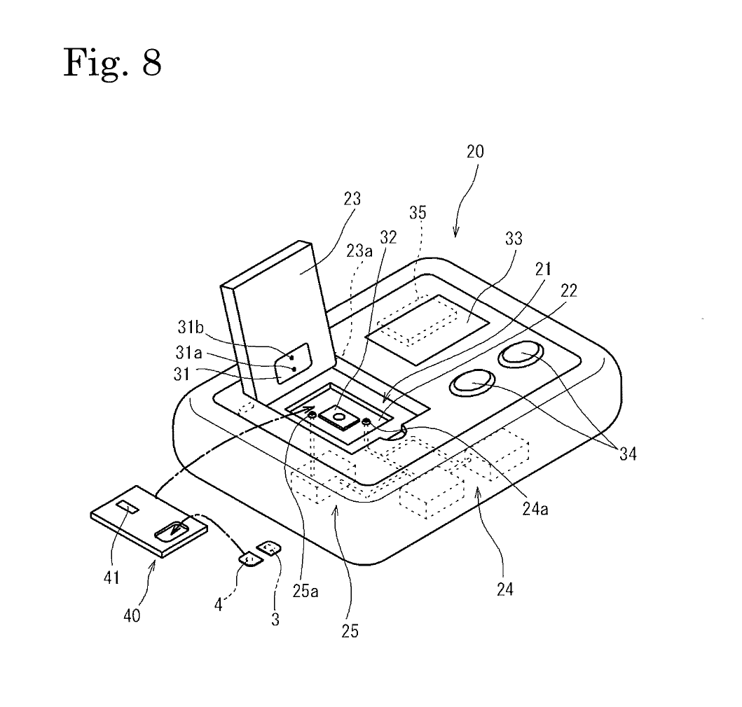

The interstitial fluid collected by the first collection body 3 is analyzed for composition, for example, using the analyzer shown in FIG. 8.

FIG. 8 is an exterior perspective view of a biological component analyzer. The biological component analyzer 20 obtains the glucose concentration and sodium ion concentration in the interstitial fluid collected by the second collection body 4. The biological component analyzer 20 is used as follows. The part including the second collection body 4 is detached from the body fluid collection device 1 which has been removed from the skin S of the subject as indicated by the dashed line in FIG. 8, and is subsequently attached to the analysis cartridge 40 which is then installed in the cartridge receiver 22 of the biological component analyzer 20. The biological component analyzer 20 executes predetermined analysis processes on the analysis cartridge 40, and the part attached thereto (including the second collection body 4), installed in the cartridge receiver 22.

The biological component analyzer 20 has a rectangular housing of some thickness, and a concavity 21 is formed in the top part of the top surface of the housing. The cartridge receiver 22 which has a receiving part that is deeper than the concavity 21 is provided in the concavity 21. A movable cover 23 which has the same thickness and height as the side wall of the concavity 21 is connected to the concavity 21. The movable cover 23 can be folded on a support shaft 23a so as to be accommodated in the concavity 21 from the state shown in FIG. 8, or may be folded open from the accommodated position in the concavity 21 to the position shown in FIG. 8. The cartridge receiver 22 has a size capable of accommodating the analysis cartridge 40.

The movable cover 23 is supported by the support shaft so as to exert a force in the direction of being accommodated in the concavity 21. The analysis cartridge 40 installed in the cartridge receiver 22 is therefore pushed from above by the movable cover 23.

The biological component analyzer 20 is internally provided with a liquid delivery unit 24 and a waste liquid unit 25. The liquid delivery unit 24 is configured to deliver a fluid to the analysis cartridge 40 installed in the cartridge receiver 22, and injects fluid into the analysis cartridge 40 installed in the cartridge receiver 22 through a nipple 24a. The waste liquid unit 25 is configured to eliminate the fluid delivered to the analysis cartridge 40 by the liquid delivery unit 24, and eliminates the fluid injected into the analysis cartridge 40 through a nipple 25a.

The biological component analyzer 20 is also provided with a glucose detection unit 31, sodium ion detection unit 32, display unit 33, operation unit 34, and controller 35.

The glucose detection unit 31 is provided on the back side of the movable cover 23, that is, on the surface of the movable cover 23 on the side opposite the cartridge receiver 22 accommodated in the concavity 21. The glucose detection unit 31 has a light source 31a which emits light, and a light receiver unit 31b which receives the reflected light among the light emitted from the light source 31a. Hence, the glucose detection unit 31 is configured to emit light to irradiate the analysis cartridge 40 installed in the cartridge receiver 22, and receive the reflected light from the irradiated analysis cartridge 40. The analysis cartridge 40 incorporates a glucose reaction body 41 which changes color through chemical reaction with glucose in the interstitial fluid collected from a living body. The glucose detection unit 31 detects the change in the degree of light absorption by the glucose based on the reflected light, and quantifies the amount of glucose from the obtained reflected light.

The sodium ion detection unit 32 is provided on the bottom surface of the cartridge receiver 22. The sodium ion detection unit 32 has a plate-like rectangular shaped member provided on the bottom surface of the cartridge receiver 22, and a pair of sodium ion concentration measurement electrodes are disposed in the approximate center of this plate-like member. The sodium ion concentration measurement electrodes include a silver/silver salt sodium ion selective electrode which has a sodium ion selective film, and a silver/silver salt counter electrode.

The controller 35 is provided in the biological component analyzer 20 and includes a CPU, ROM, RAM and the like. The CPU controls the operation of the various parts by reading and executing programs stored in the ROM. The RAM is used as the development area when executing the programs stored in the ROM.

The operation of the biological component analyzer 20 having the above mentioned structure is described below in detail.

Part (the part including the second collection body 4) of the body fluid collection device 1 which has completed collection of the interstitial fluid is loaded in the receiver of the analysis cartridge 40. The analysis cartridge 40 is then installed in the cartridge receiver 22,

When a measurement execution instruction is issued, the liquid delivery unit 24 injects the liquid through the nipple 24a into the receiver of the analysis cartridge 40. As this condition is maintained for a predetermined wait time, components in the interstitial fluid disperse from the second collection body 4 to the liquid.

After the predetermined time has elapsed, the liquid delivery unit 24 delivers air toward the receiver. When the air is delivered, the liquid filling the receiver is moved to a flow path at the bottom surface of the analysis cartridge 40, and transported to the glucose reaction body 41 through this flow path. The liquid moved through the flow path comes into contact with the sodium ion detection unit 32. The liquid supplied to the glucose detection unit 41 reacts with the glucose reaction body 41 and the glucose reaction body 41 changes color.

The controller 35 supplies a fixed voltage to the sodium ion concentration measurement electrode and obtains the current value, then obtains the sodium ion concentration based on the obtained current value and a calibration curve previously stored in the controller 35.

The controller 35 obtains the glucose concentration based on the amount of change between the amount of light received by the light receiver unit 31b before the color was produced in the color-producing agent, and the amount of light received by the light receiver unit 31b after the color was produced in the color-producing agent.

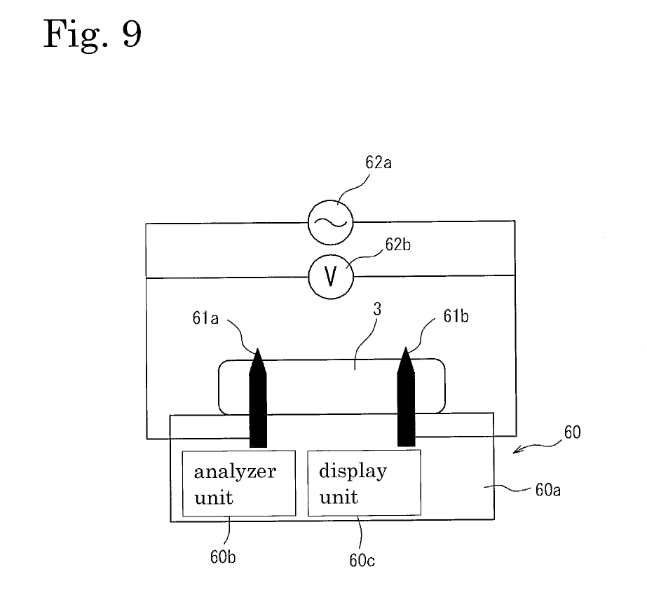

On the other hand, the perspiration collected by the first collection body 3 is measured, using a perspiration measuring device such as the one shown in FIG. 9.

FIG. 9 is a brief illustration of a perspiration measuring device used in the method for analyzing biological components of the present embodiment. The perspiration measuring device 60 has a base 60a on which the first collection body 3 is mounted, counter electrodes 61a and 61b which are disposed on the top surface of the base 60a, AC power source 62a, voltmeter 62b for measuring the current between the counter electrodes 61a and 61b, analyzer unit 60b, and display unit 60c. When the first collection body 3 is mounted on the base 60a, the counter electrodes 61a and 61b are plugged into the first collection body 3 and the counter electrodes 61a and 61b are shorted through the first collection body 3. When a voltage is supplied by the AC power source 62a in this state, the voltage between the counter electrodes 61a and 61b is measured by the voltmeter 62b. The analyzer unit 60b analyzes the sodium ion concentration collected in the first collection body 3 based on the measured voltage value and a calibration curve, and the sodium ion concentration is then shown on the display unit 60c.

The sodium ion concentration measured using the first collection body 3 can be used to improve the accuracy of the interstitial fluid measurement using the second collection body 4. For example, the measurement accuracy can be improved by comparing the measured sodium ion concentration with a predetermined threshold value so as to avoid performing the interstitial fluid measurement using the second collection body when the sodium ion concentration exceeds the threshold value, and performing the interstitial fluid measurement using the second collection body 4 when the measured sodium ion concentration is less than the threshold value. An alternative method also may be used in which the interstitial fluid measurement may be performed when the measured sodium ion concentration exceeds the threshold value, and an indication of the low reliability is displayed together with the measurement result.

### Other Embodiments

Although the first collection body 3 and the second collection body 4 are provided in the body fluid collection device 1 in the present embodiment, the body fluid collection device of the present invention is not limited to the embodiment described above. For example, the first collection body 3 and the second collection body 4 may be individually provided in different body fluid collection devices. Other embodiments of the body fluid collection device of the present invention are described below.

### Perspiration Collection Device 71

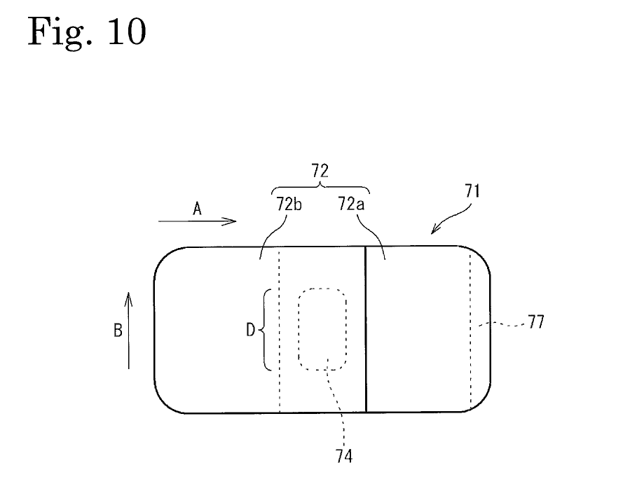

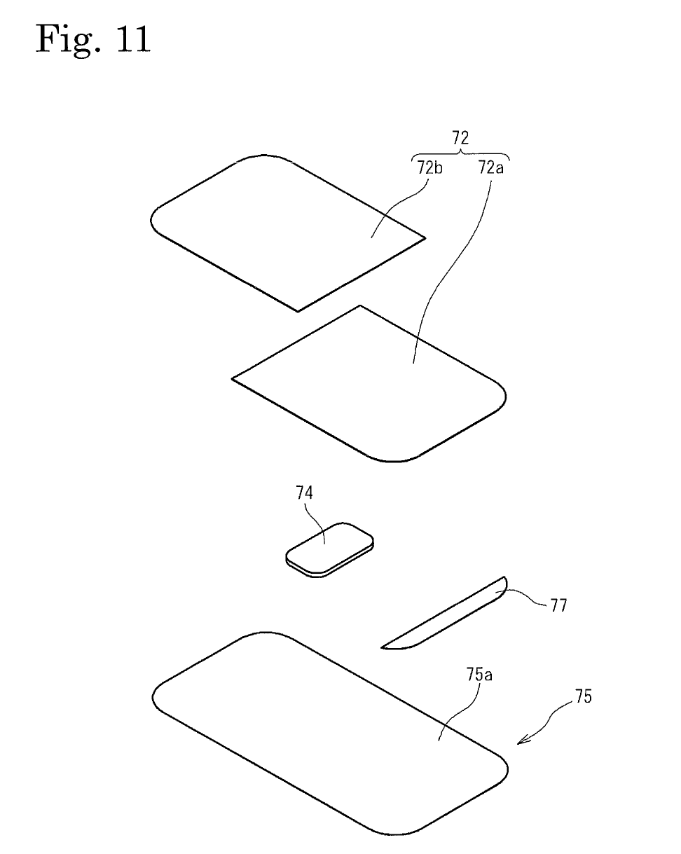

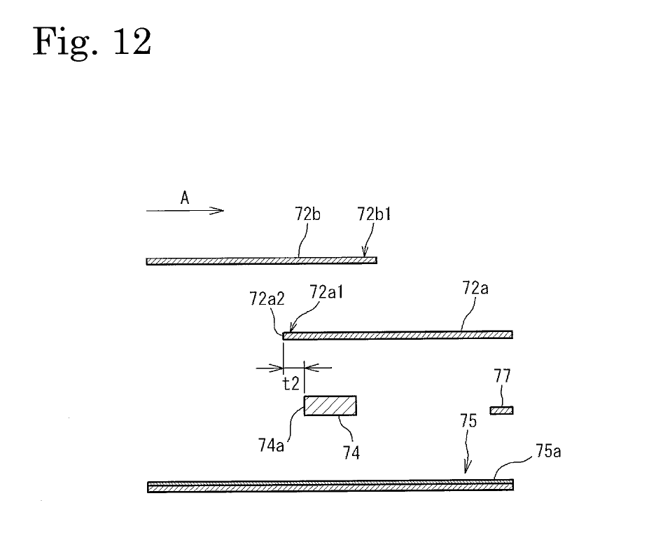

FIG. 10 is a plan view of a perspiration collection device 71 of another embodiment of the present invention, and FIG. 11 is an exploded perspective view of the perspiration collection device 71 shown in FIG. 10. FIG. 12 is a cross sectional view of the perspiration collection device 71 of FIG. 10. Note that elements such as the sheets and the like which configure the perspiration collection device 71 of the embodiment of the present invention are extremely thin members, the dimensions of which in the width direction are exaggeratedly drawn for ease of understanding in FIG. 12.

The perspiration collection device 71 is configured by a detachable sheet 72, perspiration collection body 74, and a substrate 75 sequentially from the side which is adhered to the skin S of the subject (top side in FIG. 12). Each of these elements is described below.

### Detachable Sheet

The detachable sheet 72 is configured as a two-ply sheet, that is, a first detachable sheet 72a and a second detachable sheet 72b. The first detachable sheet 72a is detachably adhered to the adhesive surface of the substrate 75 so as to cover the perspiration collection body 74. The second detachable sheet 72b is detachably adhered to the adhesive surface of the substrate 75 so as to cover the perspiration collection body 74 and part of the first detachable sheet 72a (that is, a part of the first detachable sheet 72a which includes the part that covers the perspiration collection body 74). The shape, structural material, and thickness of the first detachable sheet 72a and the second detachable sheet 72b are substantially similar to the first detachable sheet 2a and the second detachable sheet 2b. A silicone coating (silicone process) is provided on the surface to be adhered (the surface to be applied on the adhesive surface of the substrate 75) of the first detachable sheet 72a and the second detachable sheet 72b so that the detachable sheet can be removed from the adhesive surface.

### Collection Body

The perspiration collection body 74 is disposed in the approximate center on the adhesive surface of the rectangular substrate 75 which has four beveled corners. The perspiration collection body 74 is used to collect perspiration from the region of the skin S in which micropores are not formed, in the same manner as the first collection body 3 described previously.

### Substrate

The substrate 75 is used identically to the previously mentioned substrate 5.

As shown in FIGS. 10 and 12, one end 72b1 of the second detachable sheet 72b in the predetermined direction (A direction) is superimposed over the far end 72a1 of the first detachable sheet 72a so as to cover the perspiration collection body 74, in the present embodiment. Part of the far end 72a1 of the first detachable sheet 72a is separated from the adhesive surface 75a of the substrate 75 in a non-adhered state. More specifically, the far end 72a1 of the first detachable sheet 72a is not adhered completely to the entire adhesive surface 75a of the substrate 75 in the width direction (B direction in FIG. 10) of the substrate 75, and the center part D in the width direction (refer to FIG. 10) is separated from the adhesive surface 75a of the substrate 75 in a non-adhered state. In other words, the center part in the width direction of the far end 72a1 of the first detachable sheet 72a is separated from the adhesive surface 75a of the substrate 75 so as to form a space between the center part and the adhesive surface 75a. Hence, the adhesion force of the far end 72a1 of the first detachable sheet 72a relative to the adhesive surface 75a is controlled or inhibited. As a result, the first detachable sheet 72a can be easily peeled away from the substrate 75 to adhere the body fluid collection device 71 to the skin S of a subject as shall be described later.

The distance t2 between the far end 72a2 of the first detachable sheet 72a in the predetermined direction and the far end 74a of the perspiration collection body 74 is set at a distance that maintains the “non-adhered state” similar to the distance t as previously mentioned.

The knob 77 has the same structure as the previously described knob 7.

### Interstitial Fluid Collection Device 81

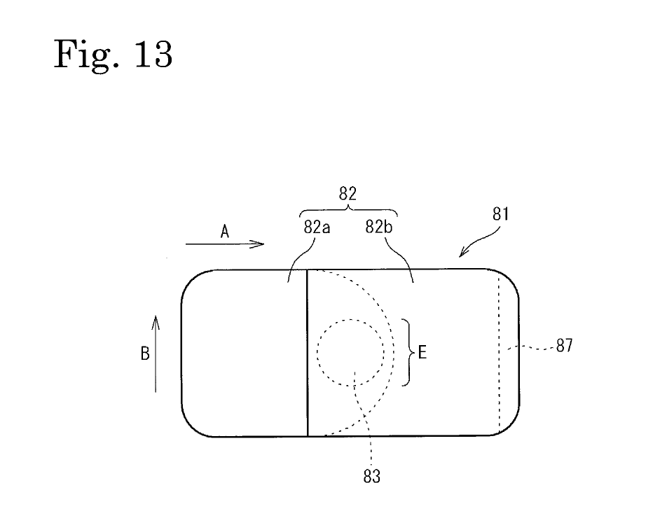

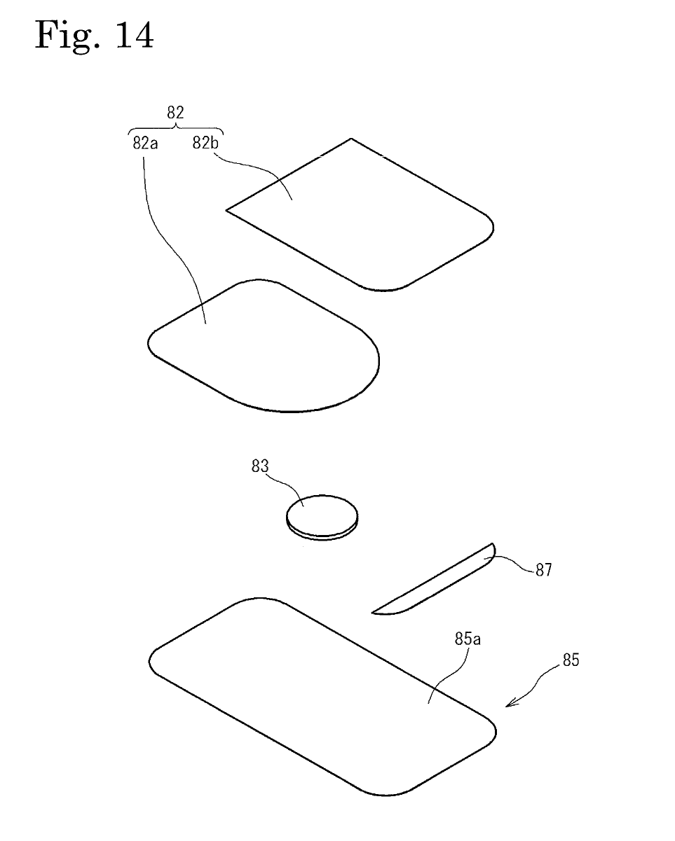

FIG. 13 is a plan view of an interstitial fluid collection device 81 of another embodiment of the present invention, and FIG. 14 is an exploded perspective view of the interstitial fluid collection device 81 shown in FIG. 13.

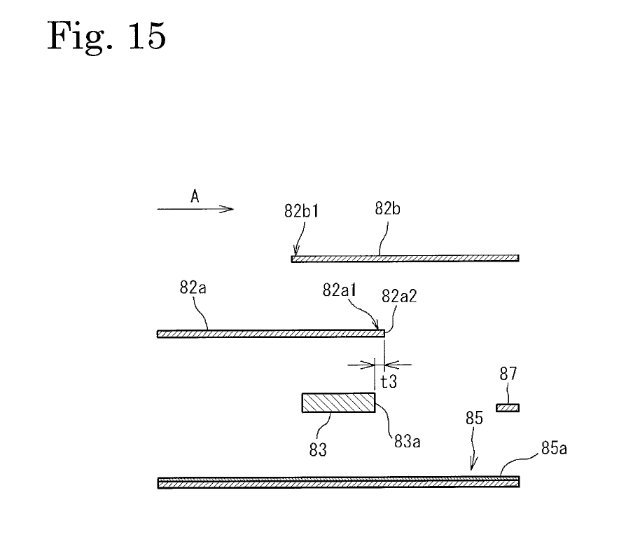

FIG. 15 is a cross sectional view of the interstitial fluid collection device 81 of FIG. 13. Note that elements such as the sheets and the like which configure the interstitial fluid collection device 81 of the present embodiment are extremely thin members, the dimensions of which in the width direction are exaggeratedly drawn for ease of understanding in FIG. 15.

The interstitial fluid collection device 81 is configured by a detachable sheet 82, interstitial fluid collection body 83, and a substrate 85 sequentially from the side which is adhered to the skin S of the subject (top side in FIG. 15). Each of these elements is described below.

### Detachable Sheet

The detachable sheet 82 is configured as a two-ply sheet, that is, a first detachable sheet 82a and a second detachable sheet 82b. The first detachable sheet 82a is detachably adhered to the adhesive surface of the substrate 85 so as to cover the interstitial fluid collection body 83. The second detachable sheet 82b is detachably adhered to the adhesive surface of the substrate 85 so as to cover the interstitial fluid collection body 83 and part of the first detachable sheet 82a (that is, a part of the first detachable sheet 82a which includes the part that covers the interstitial fluid collection body 83). The shape, structural material, and thickness of the first detachable sheet 82a and the second detachable sheet 82b are substantially similar to the first detachable sheet 2a and the second detachable sheet 2b with the exception that the far end side which covers the interstitial fluid collection body 83 of the first detachable sheet 82a has a circular shape. A silicone coating (silicone process) is provided on the surface to be adhered (the surface to be applied on the adhesive surface of the substrate 85) of the first detachable sheet 82a and the second detachable sheet 82b so that the detachable sheet can be removed from the adhesive surface.

### Collection Body

The interstitial fluid collection body 83 is disposed in the approximate center on the adhesive surface of the rectangular substrate 85 which has four beveled corners. The interstitial fluid collection body 83 is used to collect interstitial fluid from the region of the skin S in which micropores are formed, in the same manner as the second collection body 4 described previously.

### Substrate

The substrate 85 is used identically to the previously mentioned substrate 5.

As shown in FIGS. 13 and 15, one end 82b1 of the second detachable sheet 82b in the predetermined direction (A direction) is superimposed over the far end 82a1 of the first detachable sheet 82a so as to cover the interstitial fluid collection body 83, in the present embodiment. Part of the far end 82a1 of the first detachable sheet 82a is separated from the adhesive surface 85a of the substrate 85 in a non-adhered state. More specifically, the far end 82a1 of the first detachable sheet 82a is not adhered completely to the entire adhesive surface 85a of the substrate 85 in the width direction (B direction in FIG. 13) of the substrate 85, and the center part E in the width direction (refer to FIG. 13) is separated from the adhesive surface 85a of the substrate 85 in a non-adhered state. In other words, the center part in the width direction of the far end 82a1 of the first detachable sheet 82a is separated from the adhesive surface 85a of the substrate 85 so as to form a space between the center part and the adhesive surface 85a. Hence, the adhesion force of the far end 82a1 of the first detachable sheet 82a relative to the adhesive surface 85a is controlled or inhibited. As a result, the first detachable sheet 82a can be easily peeled away from the substrate 85 to adhere the interstitial fluid collection device 81 to the skin S of a subject as shall be described later.

The distance t3 between the one end 82a2 of the first detachable sheet 82a in the predetermined direction and the one end 83a of the interstitial fluid collection body 83 in the predetermined direction is set at a distance that maintains the “non-adhered state” similar to the distance t as previously mentioned.

The knob 87 has the same structure as the previously described knob 7.

### Method of Adhering the Perspiration Collection Device 71

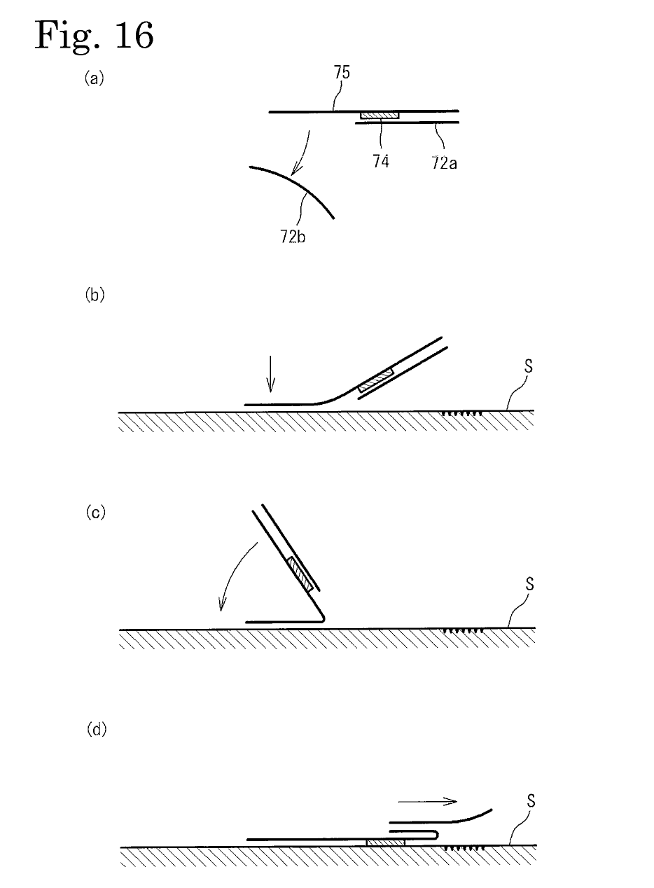

FIG. 16 illustrates the procedure of adhering the perspiration collection device 71 on the skin S of a subject. The second detachable sheet 72b is first peeled from the substrate 75 (procedure (a)). The adhesive surface 75a of the substrate 75 which was exposed by peeling away the second detachable sheet 72b is then adhered to the skin S of the subject (procedure (b)). At this time the attachment position of the adhesive surface 75a is adjusted so that the perspiration collection body 74 is positioned near the region in which micropores are formed in the skin S of the subject. Thereafter, the first detachable sheet 72a is folded over on the adhesion position side (procedure (c)), the first detachable sheet 72a is slid from the folded state so as to press on the skin S of the subject and gradually adhere the adhesive surface 75a which was covered by the first detachable sheet 72a on the skin S of the subject (procedure (d)). The perspiration collection device 71 is adhered to the skin S of the subject through this sequence of procedures.

### Method of Adhering the Interstitial Fluid Collection Device 81

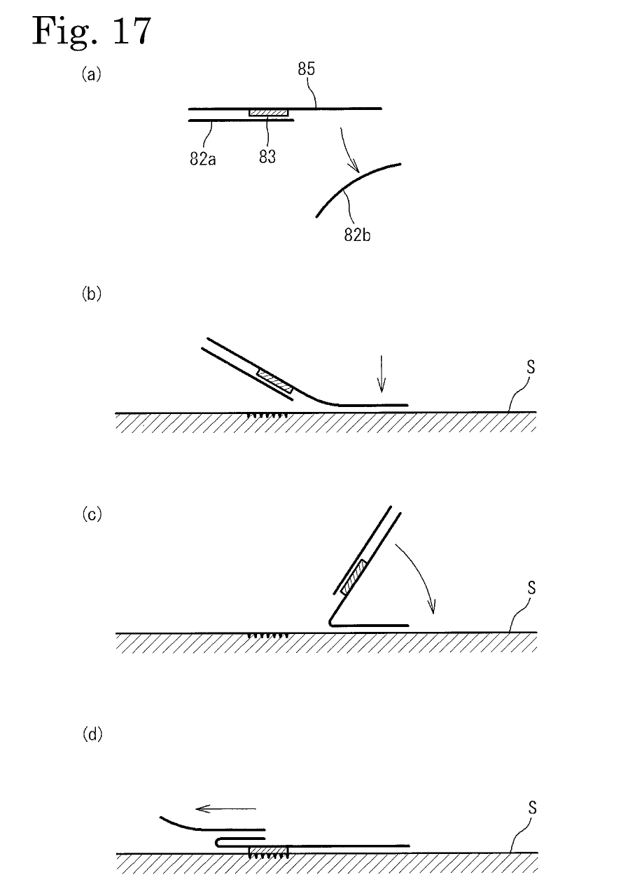

FIG. 17 illustrates the procedure of adhering the interstitial fluid collection device 81 on the skin S of a subject. The second detachable sheet 82b is first peeled from the substrate 85 (procedure (a)). The adhesive surface 85a of the substrate 85 which was exposed by peeling away the second detachable sheet 82b is then adhered to the skin S of the subject (procedure (b)). At this time the attachment position of the adhesive surface 85a is adjusted so that the interstitial fluid collection body 83 is positioned in the region in which micropores were formed in the skin S of the subject and the entire interstitial fluid collection device 81 is adhered. Thereafter, the first detachable sheet 82a is folded over on the adhesion position side (procedure (c)), the first detachable sheet 82a is slid from the folded state so as to press on the skin S of the subject and gradually adhere the adhesive surface 85a which was covered by the first detachable sheet 82a on the skin S of the subject (procedure (d)). The interstitial fluid collection device 81 is adhered to the skin S of the subject through this sequence of procedures.

In the body fluid collection device 1, the detachable sheet is formed by two layers of detachable sheets (first detachable sheet, second detachable sheet), and one end of the second detachable sheet is overlaid on the far end of the first detachable sheet. Part of the far end of the first detachable sheet is separated from the adhesive surface of the substrate in a non-adhered state. When in use, the second detachable sheet therefore is first peeled off to adhere the exposed adhesive surface to the skin, then the first detachable sheet is folded back and the first detachable sheet is slid along the skin surface to easily adhere the body fluid collection device on the skin of the subject without touching the collection body. In this case, in the body fluid collection device 1, part of the far end of the first detachable sheet is separated from the adhesive surface and in a non-adhered state, so that the operation of sliding the first detachable sheet proceeds smoothly after the first detachable sheet has been folded over because there is only slight adhesion between the first detachable sheet and the adhesive surface.

### Modifications

Note that the present invention is not limited to the above described embodiments and may be variously modified insofar as such modification are within the scope of the claims.

For example, although the knob is provided at the end of the substrate in the longitudinal direction in the above embodiments, the knob also may be omitted.

In the above embodiments, the first collection body 3 collects perspiration from the region of the skin S in which micropores are not formed and the second collection body 4 collects interstitial fluid from the region in which micropores are formed, however, this arrangement also may be reversed.

What is claimed is:

1. A body fluid collection device for collecting body fluid through the skin, comprising:
a sheet-like substrate provided with an adhesive surface;
a first collection body for collecting body fluid, provided on the adhesive surface;
a first detachable sheet which is detachably adhered on the adhesive surface so as to cover the first collection body; and
a second detachable sheet which is detachably adhered on the adhesive surface, wherein
one end of the second detachable sheet in a predetermined direction is overlaid on the far end of the first detachable sheet in the predetermined direction, and
part of the far end of the first detachable sheet is separated from the adhesive surface in a non-adhered state.

2. The body fluid collection device of claim 1, wherein a space is formed between a part of the far end of the first detachable sheet and the adhesive surface when the second detachable sheet is peeled from the substrate.

3. The body fluid collection device of claim 1, wherein the distance between the far end of the first detachable sheet in the predetermined direction and the far end of the first collection body in the predetermined direction is less than the distance L defined by equation (1) below.
L=(2.4/0.7)×d (1)
(Where d is the thickness of the first collection body.)

4. The body fluid collection device of claim 1, wherein the distance between the far end of the first detachable sheet in the predetermined direction and the far end of the first collection body in the predetermined direction is 1 mm or greater but less than 3 mm.

5. The body fluid collection device of claim 1, wherein the external shape of the first collection body is substantially cuboidal or substantially rectangular.

6. The body fluid collection device of claim 1, wherein the first collection body is a gel-like member.

7. The body fluid collection device of claim 1, wherein the second detachable sheet is adhered to the adhesive surface so as to cover part of the first detachable sheet which includes the part covering the first collection body.

8. The body fluid collection device of claim 1 further comprising a second collection body for collecting body fluid provided on the adhesive surface;
the first collection body and the second collection body are provided so as to be mutually separated along the predetermined direction on the adhesive surface.
the second detachable sheet is adhered to the adhesive surface so as to cover the second collection body.

9. The body fluid collection device of claim 8, wherein the second collection body is a gel-like member.

10. The body fluid collection device of claim 8, wherein the first collection body collects perspiration from a region on the skin in which micropores have not been formed, and the second collection body collects interstitial fluid from the region of the skin in which micropores have been formed.

11. The body fluid collection device of claim 8, wherein the distance between the far end of the first collection body in the predetermined direction and the far end of the second collection body in the predetermined direction is 10 mm or greater but less than 20 mm.

12. The body fluid collection device of claim 8, wherein surfaces to be applied on the adhesive surface of the first detachable sheet and the second detachable sheet are treated with a silicone process.

13. The body fluid collection device of claim 8, wherein the second collection body has a different shape than the first collection body.

14. The body fluid collection device of claim 1, wherein the first detachable sheet has a thickness of 0.025 mm or greater but less than 0.100 mm.

15. The body fluid collection device of claim 1, wherein the first detachable sheet is made of a synthetic resin containing at least one in a group of polyethylene terephthalate and polyolefin.

16. The body fluid collection device of claim 1, wherein the substrate has a thickness of 0.025 mm or greater but less than 0.100 mm.

17. The body fluid collection device of claim 1, wherein the substrate is made of a synthetic resin containing at least one in a group of polyethylene, polypropylene, polyester, and polyurethane.

18. The body fluid collection device of claim 1, wherein the first collection body has a thickness of 0.05 mm or greater but less than 1.5 mm.

19. A body fluid collection device for collecting body fluid through the skin, comprising:
a sheet-like substrate provided with an adhesive surface;
a first collection body for collecting body fluid, provided on the adhesive surface;
a first detachable sheet which is detachably adhered on the adhesive surface so as to cover the first collection body; and
a second detachable sheet which is detachably adhered on the adhesive surface, wherein
one end of the second detachable sheet in a predetermined direction is overlaid on the far end of the first detachable sheet in the predetermined direction,
the first collection body has a thickness of 0.05 mm or greater but less than 1.5 mm,
the first detachable sheet has a thickness of 0.025 mm or greater but less than 0.100 mm, and is made of at least one in a group of polyethylene terephthalate and polyolefin, and
the distance between the far end of the first detachable sheet in the predetermined direction and the far end of the first collection body in the predetermined direction is 1 mm or greater but less than 3 mm.

20. A body fluid collection device for collecting body fluid through the skin, comprising:
a sheet-like substrate provided with an adhesive surface;
a first collection body for collecting body fluid, provided on the adhesive surface;
a second collection body for collecting body fluid, provided on the adhesive surface;
a first detachable sheet which is detachably adhered on the adhesive surface so as to cover the first collection body; and
a second detachable sheet which is detachably adhered on the adhesive surface so as to cover the second collection body; wherein
the first collection body and the second collection body are provided so as to be mutually separated along the predetermined direction on the adhesive surface, and
one end of the second detachable sheet in a predetermined direction is overlaid on the far end of the first detachable sheet in the predetermined direction, that is, part of the far end of the first detachable sheet is separated from the adhesive surface in a non-adhered state.
